(19) United States
(12) Patent Application Publication
(10) Pub. No.: US 2025/0076029 A1
(43) Pub. Date: Mar. 6, 2025

(54) FRUSTRATED TOTAL INTERNAL REFLECTION (FTIR) SURFACE TOPOGRAPHY AND COMPOSITION ANALYSIS SYSTEMS, METHODS, AND DEVICES

(71) Applicant: GS-HealthMatrix, LLC, Los Angeles, CA (US)

(72) Inventors: Joseph Schwab, Los Angeles, CA (US); Amirhossein Yazdkhasti, Los Angeles, CA (US); Hamid Ghaednia, Los Angeles, CA (US)

(73) Assignee: GS-HealthMatrix, LLC, Los Angeles, CA (US)

(21) Appl. No.: 18/819,148

(22) Filed: Aug. 29, 2024

Related U.S. Application Data
(60) Provisional application No. 63/579,605, filed on Aug. 30, 2023.
(60) Provisional application No. 63/579,616, filed on Aug. 30, 2023.
(60) Provisional application No. 63/579,627, filed on Aug. 30, 2023.
(60) Provisional application No. 63/579,633, filed on Aug. 30, 2023.
(60) Provisional application No. 63/579,640, filed on Aug. 30, 2023.
(60) Provisional application No. 63/579,647, filed on Aug. 30, 2023.
(60) Provisional application No. 63/579,663, filed on Aug. 30, 2023.

(51) Int. Cl. G01B 11/24; A61B 5/00; G01N 21/55

(52) U.S. Cl. CPC G01B 11/24; A61B 5/0062; G01N 21/55

(57) ABSTRACT

Systems, methods, and devices include a frustrated total internal refraction (FTIR) based scanning device. The FTIR based scanning device has a transparent media and one or more electromagnetic wave emitters operable to provide a scanning light into the transparent media during a sample scanning procedure. One or more electromagnetic wave sensors, cameras, and/or microscopes are directed at a detection surface of the transparent media. These detection component(s) receive scattered light passing from the sample contact surface through the detection surface. The device uses the scattered light to represent a surface topology or a material composition of a sample contacting the sample contact surface during the sample scanning procedure. Additionally, the one or more electromagnetic wave emitters can include a plurality of LEDs or electromagnetic wave emitters corresponding to a plurality of different wavelengths which are used to generate an image of a 3D topology from the scattered light.

### CROSS-REFERENCE TO RELATED APPLICATIONS

This application claims priority to U.S. Provisional Application Ser. No. 63/579,605 filed Aug. 30, 2023 and titled “FRUSTRATED TOTAL INTERNAL REFLECTION (FTIR) SURFACE TOPOGRAPHY AND COMPOSITION ANALYSIS SYSTEMS, METHODS, AND DEVICES;” U.S. Provisional Application Ser. No. 63/579,616 filed Aug. 30, 2023 and titled “SYSTEMS, METHODS, AND DEVICES OF WEARABLE ELECTRO-ACOUSTIC MONITORING;” U.S. Provisional Application Ser. No. 63/579,627 filed Aug. 30, 2023 and titled “SYSTEMS, METHODS, AND DEVICES FOR ACOUSTICALLY ENHANCING IMPLANTS;” U.S. Provisional Application Ser. No. 63/579,633 filed Aug. 30, 2023 and titled SYSTEMS, METHODS, AND DEVICES WITH SENSORS HAVING MULTIPLE DETECTION SIGNAL TYPES;” U.S. Provisional Application Ser. No. 63/579,640 filed Aug. 30, 2023 and titled MULTI-DEVICE HEALTH PARAMETER MONITORING SYSTEMS, METHODS, AND DEVICES;” U.S. Provisional Application Ser. No. 63/579,647 filed Aug. 30, 2023 and titled FRUSTRATED TOTAL INTERNAL REFLECTION (FTIR)-BASED HEALTH PARAMETER DETECTION SYSTEMS, METHODS, AND DEVICES;” and U.S. Provisional Application Ser. No. 63/579,663 filed Aug. 30, 2023 and titled “SYSTEMS, METHODS, AND DEVICES FOR NEUROLOGICAL AND/OR MUSCOSKELETAL PARAMETER CHARACTERIZATION;” the entireties of which are herein incorporated by reference.

### BACKGROUND

Three-dimensional (3D) topography measurements typically use a laser or a mechanical stylus to measure a surface and construct 3D maps of the surface. None of these measurement techniques are capable of generating 3D maps of surfaces of objects that are in direct contact. A measurement distance is conventionally required between the measuring device and the surface. There are no known methods for creating 3D topography at the contact interface because the surface at the contact interface is not visible to the testing equipment. Conventional techniques are also very slow.

It is with these observations in mind, among others, that various aspects of the present disclosure were conceived and developed.

### SUMMARY

The systems, methods, and devices disclosed herein can address the aforementioned issues. For instance, a frustrated total internal refraction (FTIR) based scanning device can include a transparent media having a sample contact surface; one or more electromagnetic wave emitters operable to provide a scanning light into the transparent media during a sample scanning procedure; and/or one or more electromagnetic wave sensors, cameras, or microscopes. The one or more electromagnetic wave sensors, cameras, or microscopes can be directed at a detection surface of the transparent media. Additionally, the one or more electromagnetic wave sensors, cameras, or microscopes can be operable to receive scattered light passing from the sample contact surface through the detection surface. Moreover, the device can use the scattered light to represent a surface topology or a material composition of a sample contacting the sample contact surface during the sample scanning procedure.

In some instances, the one or more electromagnetic wave emitters include a plurality of LEDs or electromagnetic wave emitters corresponding to a plurality of different wavelengths. Additionally, providing the scanning light can include individually illuminating the plurality of LEDS to scan the sample with a sequence of different frequencies. The transparent media can include a glass sheet, and/or the one or more electromagnetic wave emitters can be positioned at one or more side surfaces of the glass sheet for transmitting the scanning light into the glass sheet. Furthermore, the glass sheet can be a flat glass sheet or a curved glass sheet. Additionally, the sample contact surface can include a raised portion operable to indent the sample during the sample scanning procedure. The transparent media can be formed into a handheld device with the sample contact surface defining an end of the handheld device, and/or the one or more electromagnetic wave sensors, cameras, or microscopes can be disposed in an interior portion of the handheld device. The device can also include a computing device having at least a display operable for presenting an image of a 3D topology generated from the scattered light. Also, the one or more electromagnetic wave sensors, cameras, or microscopes can include one or more of an infrared camera, a visible light camera, or an ultraviolet light camera.

In some examples, a method to perform a surface topology or composition analysis includes contacting at least a portion of a sample with a first surface of a transparent media of a scanning device; providing a scanning light into the transparent media by activating one or more LEDs or electromagnetic wave emitters; receiving, at one or more light sensors of the scanning device, scattered light resulting from a force on the first surface of the transparent media caused by at least the portion of the sample, the scattered light passing out a second surface of the transparent media to reach the one or more light sensors; and/or generating, based on the scattered light received at the one or more light sensors, a surface topology or a material composition of at least the portion of the sample.

In some scenarios, the one or more LEDs or electromagnetic wave emitters can include a plurality of different frequency LEDs or electromagnetic wave emitters; and/or generating the surface topology or the material composition can include aggregating different frequencies of the scattered light, generated one-by-one by the plurality of different frequency LEDs or electromagnetic wave emitters into a profilometry for at least the portion of the sample contacting the first surface. The one or more light sensors can include at least one of an infrared camera, a visible light camera, or an ultraviolet light camera disposed in the scanning device and directed at the transparent media. Also, providing the scanning light into the transparent media can include activating a plurality of LEDs or electromagnetic wave emitters positioned adjacent to a third surface of the transparent media. Additionally, the transparent media can include a glass sheet and the first surface can be an exposed top surface of the glass sheet. Furthermore, the second surface can be an unexposed bottom surface of the glass sheet opposite the exposed top surface; and/or the third surface can be a side surface of the glass sheet. The force on the first surface of the transparent media can be caused by at least the portion of the sample creating the scattered light by using frustrated total internal refraction (FTIR).

In some instances, a system for generating a surface topology or composition analysis includes a transparent media having a sample contact surface; a plurality of LEDs with different frequencies operable to provide scanning light into a side of the transparent media during a sample scanning procedure; one or more light sensors, directed at a detection surface of the transparent media and operable to receive scattered light resulting from a force at the sample contact surface; and/or a surface topology or a material composition of a sample contacting the sample contact surface during the sample scanning procedure, the surface topology or the material composition being generated from the scattered light.

In some examples, the system can include a profilometry of a portion of the sample contacting the sample contact surface during the sample scanning procedure, the profilometry including an aggregation of different frequencies of the scattered light, the surface topology being a three-dimensional representation of the profilometry. Furthermore, the system can be integrated into a handheld scanning device or a standing platform. The system can also include the material composition; and/or the different frequencies can be selectively activated to correspond to a target component of the material composition. Also, the sample can include a living body part of a human, a living body part of an animal, or a plant; and/or the surface topology can include a tumor surface topography, a human organ surface topology, or a plant leaf surface topology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIGS. 1A and 1B illustrate an example system for generating a surface topology or a composition analysis using a frustrated total internal refraction (FTIR) based scanning device.

FIG. 1C illustrates an example profilometry of a system for generating a surface topology or a composition analysis using an FTIR based scanning device.

FIGS. 2A-2C illustrate example form factors of an FTIR based scanning device for generating a surface topology or a composition analysis for different contact mechanic testing scenarios where the devices could be used to analyze different modes of deformation of soft and hard objects.

FIGS. 3A-3C illustrate an example FTIR based scanning device for generating a surface topology or a composition analysis of a plant.

FIGS. 4A and 4B illustrate an example FTIR based scanning device for generating a surface topology or a composition analysis for skin health monitoring.

FIGS. 5A and 5B illustrate an example FTIR based scanning device for generating a surface topology or a composition analysis for internal medicine.

FIG. 6 illustrates an example method of generating a surface topology or a composition analysis using a frustrated total internal refraction (FTIR) based scanning device, which can be implemented by any of the systems and devices depicted in FIGS. 1-5B.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “example,” and/or the like in the description mean that the feature and/or features being referred to are included in, at least, one aspect of the description. Separate references to the terms “embodiments,” “examples,” and/or the like in the description do not necessarily refer to the same example and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments and examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein use the concept of Frustrated Total Internal reflection (FTIR) for 3D scanning of a surface topography at the interface of contact between the scanning device and the surface topography. The scanning device disclosed herein can determine the material/chemical composition of the contacting object simultaneously with generating a virtual representation of the surface topography. During operation, the scanning device can trap electromagnetic waves (e.g., ultraviolet, visible, and infrared lights) in a transparent medium. This can be done using several different LEDs for each different wavelength of light. In these conditions, an electromagnetic field can be created around the boundary of the transparent object that keeps the photons trapped inside the transparent medium. If any object gets closer to the surface than the wavelength distance of the trapped electromagnetic wave, photons start scattering from the object and through the transparent medium. A camera on the other side of the transparent medium can detect these scattered photons.

Accordingly, in some instances, the scanning device can use a glass sheet to trap electromagnetic waves while being pressed against an object being measured. A camera on the other side of the glass opposite from the object being measured can record the scattered photons. The LEDs with different wavelengths can be turned on one-by-one (e.g., in a sequence), for instance, from smaller wavelengths to larger wavelengths, and/or in various combinations of frequencies (e.g., all at once simultaneously). The camera can record the scattered photos at the different LED illumination stages to record slices of the contacting area at different wavelengths corresponding to distances from the glass surface. Because the different distances from the glass surface correspond to the different wavelengths of the LEDs, by combining these different wavelength pictures together, a 3D surface topography based on the corresponding distances to the contact area can be generated.

Moreover, the technology disclosed herein can provide information about the material composition of the contacting material. FTIR spectroscopy for determining the material can be performed simultaneously with generating the 3D surface topology. One or more cameras (e.g., instead of light spectrometers) can detect electromagnetic waves emitted one by one and only at the wavelengths needed for the material composition analysis. For example, if the scanning device is used to check the oxygen level of a material, LEDs that emit certain wavelengths of EM waves specifically for detecting oxygen can be used (e.g., between 1400-1600 nm). In some examples, the chemical composition analysis performed with a camera and multiple different wavelengths is an improvement over other FTIR spectroscopy devices that may use only light spectrometers.

Many benefits and advantages can result from the presently disclosed technology. The scanning device can be used as a measurement instrument in the field of tribology to improve our understanding of interfaces between objects. This can be especially helpful for rough surface contact and the friction between objects, which are used in several industries such as automobile, sub-sea, energy, etc. Furthermore, the technology can be used in endoscopy where it can be used to look at the surface topography of tumors or organs and help with easier diagnosis, progress assessment, evaluation, and treatment. Other benefits of the scanning device could be realized with a handheld or portable device to check surface topography of leaves. This could help with optimization of pesticides, evaluation of leaf wettability, and plant health. Furthermore, the systems, methods, and devices disclosed herein can be used for skin health monitoring and/or detection of malignant skin lesions. Benefits can also result by inclusion of this technology into current measurement instruments such as the Bruker mechanical testing systems, or nano indentation devices. Moreover, some instances of the scanning device, as described herein, can be used in agriculture and medicine.

Additional advantages of the systems, methods, and devices discussed herein will become apparent from the detailed description below.

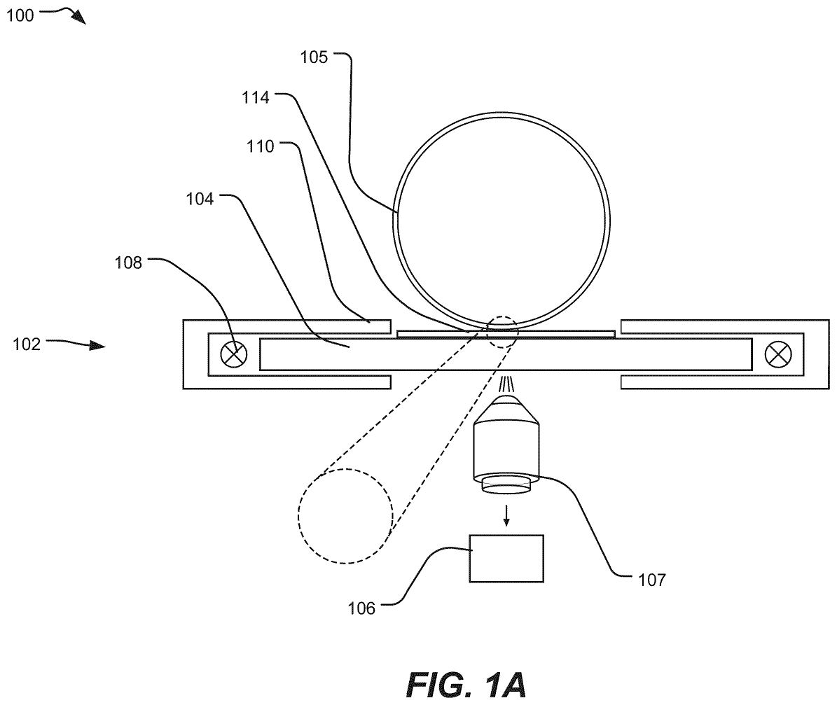

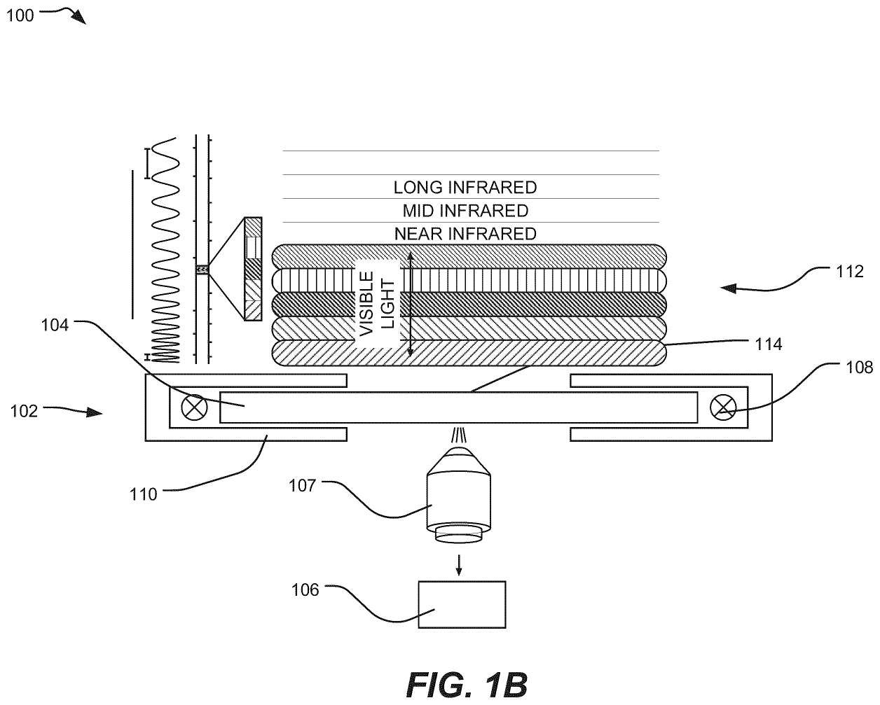

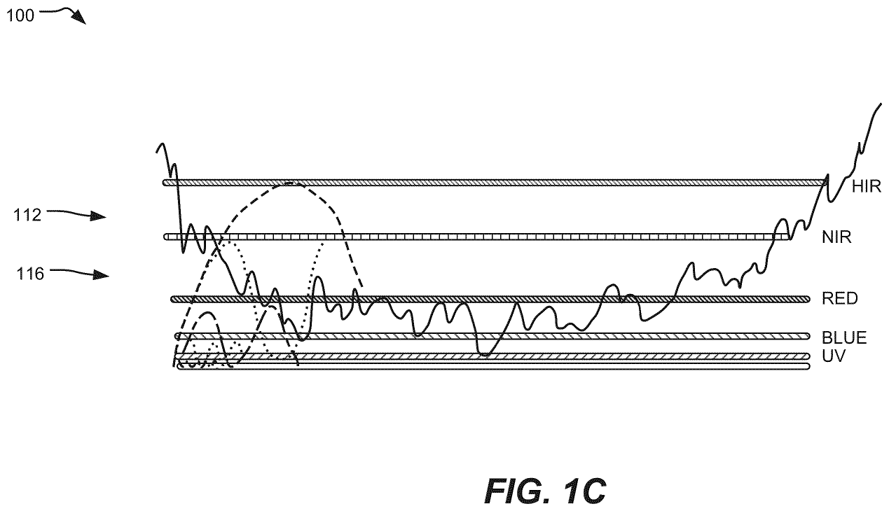

FIGS. 1A-1C illustrate an example system 100 for generating a surface topology or a material composition including an FTIR based scanning device 102. The FTIR based scanning device 102 can use the concept of FTIR to trap different wavelengths of electromagnetic waves inside a transparent media (e.g., glass portion 104). In this condition, if any object (e.g., a sample 105) gets closer to the glass medium than the wavelength of the trapped light, photons start scattering from the contacting areas. Therefore, one or more cameras 106, light sensors, and/or a microscope 107 under the glass medium could record those photons. In some instances, different wavelengths of electromagnetic waves from ultraviolet range to visible and infrared waves are generated by one or more LEDs (e.g., a plurality of LEDs 108). The plurality of LEDs 108 can include a wide range of LEDs, for instance, between a 10 nm wavelength and a 4000 nm wavelength. The plurality of LEDs can also be arranged in a strip or array inside the FTIR based scanning device 102. Moreover, the FTIR based scanning device 102 can include one or more interchangeable LED assemblies that correspond to a particular use case, a particular component of the material composition, and so forth. The FTIR based scanning device 102 can trap and record disturbances to these waves one-by-one to create images of the scattered photons. The glass medium can also have an electrically conductive portion, such as transparent, electrically conducting ink, formed onto the contact surface of the glass portion 104. Furthermore, other portions of the glass portion 104 can be covered with a shield 110 or an opaque material to trap the photons in the glass portion 104

The FTIR based scanning device 102 can be used to generate images of different slices of the object close to the glass medium. As such, the FTIR based scanning device 102 can operate as a nano scale 3D scanner which constructs the 3D profile of the surface of the contacting area. Moreover, by analyzing the amplitude of the receiving wave for each of these wavelengths the FTIR based scanning device 102 can simultaneously determine the material composition of the contacting object. Accordingly, the FTIR based scanning device 102 can improve the user's fundamental understanding of friction with direct impacts on automobile, energy, subsea, electronic, and healthcare industries.

FIG. 1B depicts a diagram of the different wavelengths used for measuring a surface topography. As discussed above, the plurality of LEDs 108 can correspond to a plurality of different wavelengths 112 used to create the 3D surface topology and/or the material composition. The plurality of different wavelengths 112 can each define a measurement distance from a contact surface 114 of the glass portion 104, wherein an object coming within the measurement distance causes the photons to scatter for that particular wavelength. FIG. 1C depicts an example profilometry 116 of a rough surface contact. FIG. 1C shows how FTIR can be used to measure the rough surface of the contacting area. This could be used for development of handheld and portable profilometers that are used in several different fields and industries.

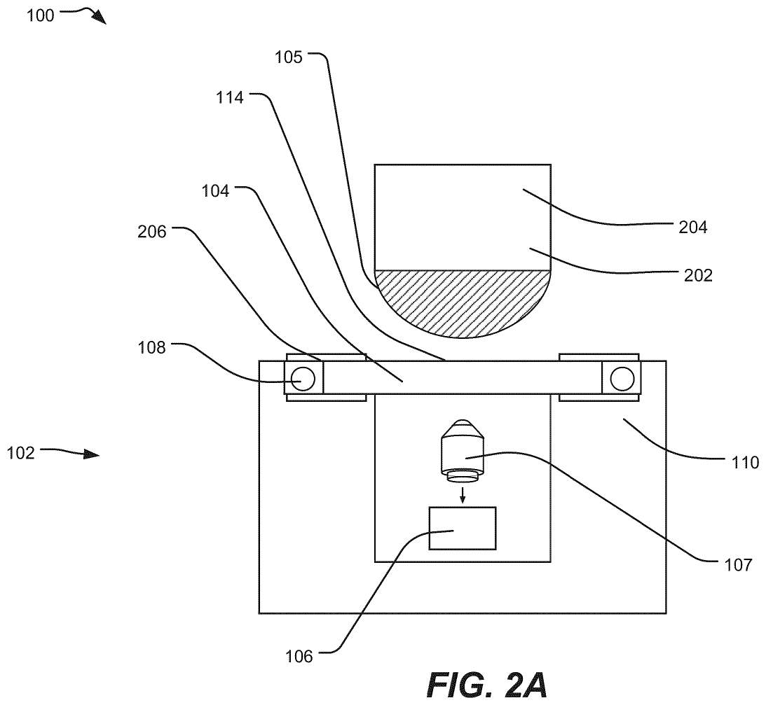

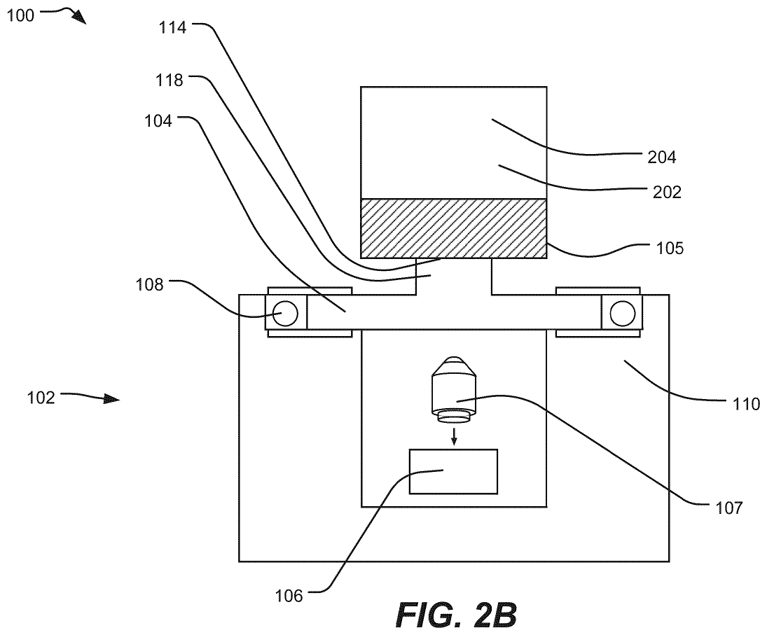

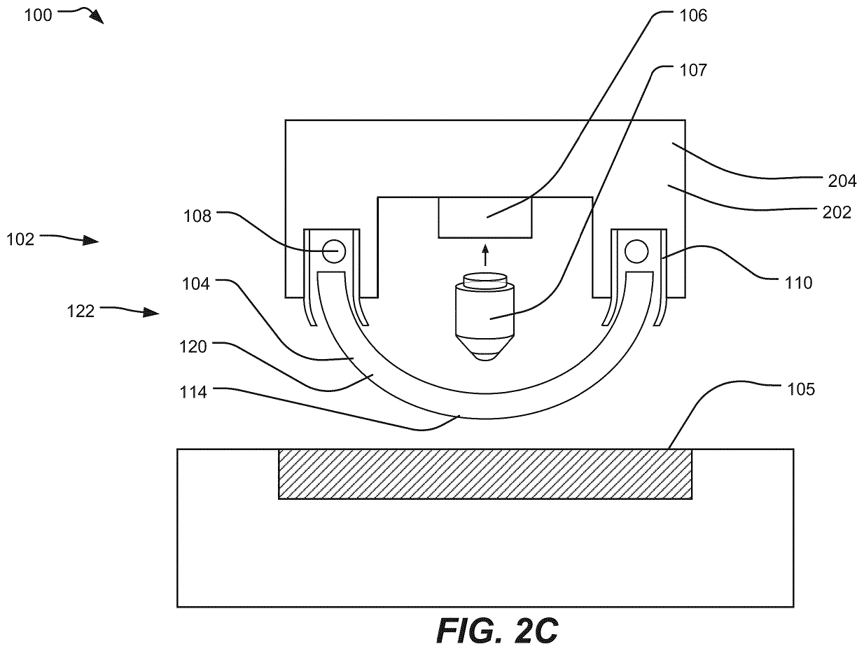

FIGS. 2A-2C depict example contact form factors of the FTIR based scanning device 102. In some instances, the FTIR based scanning device 102 can form a part of a measurement devices used in contact mechanics, such as indenters that are used for understanding contact of different surfaces. For example, the FTIR based scanning device 102 can be used for optimization of interfaces, for example, in ball bearings used in wind turbines. Additionally, the FTIR based scanning device 102 can be used for material properties measurement, better understanding of concepts of contact mechanics, optimization of electrical contacts such as chargers of smart phones or faster USB cables, and so forth. Some of the different types of contact mechanic tests that can benefit from this technology are depicted in FIGS. 2A-2C.

FIG. 2A depicts an example of the FTIR based scanning device 102 with a flat contact surface 114 in which the sample 105 can be a conical, cylindrical, spherical, or a flat punch that is pressed against the contact surface 114 of the glass portion 104 (e.g., the transparent sheet of sapphire glass). Additionally, the FTIR based scanning device can include a force transducer 202 and/or fixture and displacement sensor 204 which can also make contact with the sample 105 (e.g., on a side of the sample 105 opposite from the contact surface 114. Furthermore, the FTIR based scanning device 102 can include a temperature controller 206 (e.g., a heat sink, a heating/cooling surface, and/or an insulation component) which controls a surface temperature of the contact surface 114.

FIG. 2B depicts an example of the FTIR based scanning device 102 with a contact surface 114 having a raised contact nest 118. The sample 105 can be soft and/or flat. As shown in FIG. 2B, the contact surface of the FTIR based scanning device 102 can have a stepped or raised portion shaped into a punch indenter for indenting the sample 105 during the scanning procedure.

FIG. 2C depicts an example of the FTIR based scanning device 102 having a non-flat contact surface 120 (e.g., curved, spherical, slanted, etc.) such that the contact surface 114 is an indenter 122 of a round shape or any other shape when the contact surface contacts/indents the sample. In other words, the glass itself could be used as an indentation tip that penetrates the objects as well as a hard flat surface that contacts the object. In contact mechanics, these two scenarios can be called indentation contact and flattening contact, respectively.

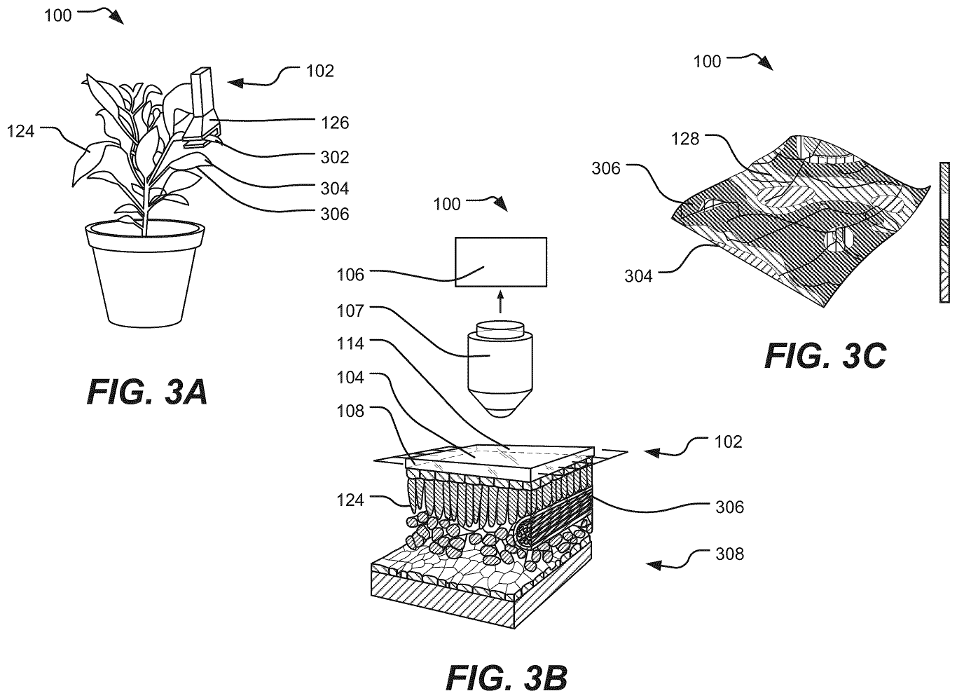

FIGS. 3A-3C depict an example of the FTIR based scanning device 102 for health monitoring of plants 124 via measuring surface topography 302 of their leaves 304. As shown in FIG. 3A, the FTIR based scanning device 102 can be a handheld device 126 designed to be pressed onto a leaf 304 of a plant 124 and to perform measurements of the surface 306 of the plant 124. The FTIR based scanning device 102 can include two contact surfaces connected at one side of the FTIR based scanning device 102 forming a receiving gap at the other side of the FTIR based scanning device 102. FIG. 3B depicts a sectional view 308 of the leaf 304 positioned between a first contact surface of the FTIR based scanning device 102 and a second contact surface of the FTIR based scanning device 102. The two contact surfaces can be flat or curved. One or both of the contact surfaces can include a glass portion 104 for performing the FTIR based measurements. Furthermore, FIG. 3C shows an example three-dimensional profile 128 (e.g., of the leaf surface 306) which can be generated by the FTIR based scanning device 102. The surface topography of plants 124 can be used for optimization of pesticides, measurement of the leaf wettability, diagnosis of diseases, and so forth.

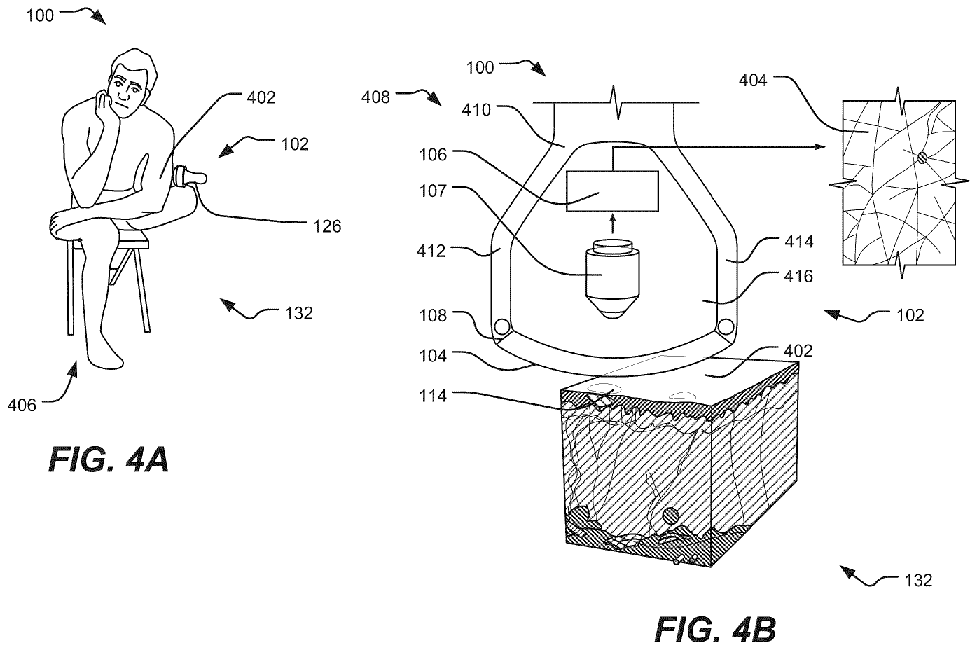

FIGS. 4A and 4B depict an example of the FTIR based scanning device 102 for skin health monitoring 132. For instance, as, shown in FIG. 4A, the FTIR based scanning device 102 can have a curved (e.g., or flat) contact surface that can be pressed into the skin 402 at a target area 404 of a patient 406. FIG. 4B shows the sensing head 408 of the FTIR based scanning device 102 pushed against the skin 402. The sensing head 408 can include the contact surface mounted to a handle 410, for instance at a widened portion 412 of the handle 410. The widened 412 portion can include an outer wall 414 disposed around an inner sensing cavity 416.

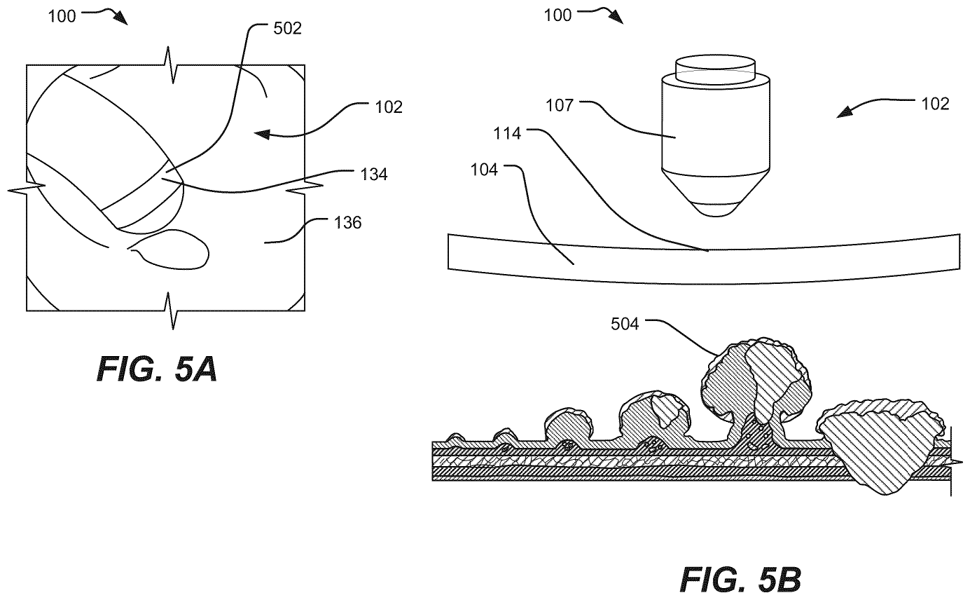

FIGS. 5A and 5B depict an example of the FTIR based scanning device 102 integrated into an endoscopy probe 134. For instance, as shown in FIG. 5A, the FTIR based scanning device 102 can form an end portion 502 of the endoscopy probe 134 which can be used to help diagnose conditions in a colon 136, such as characteristics of a tumor 504. The FTIR based scanning device 102 can also be formed into an arthroscopy for measuring, for example, the health condition of joints, bones, cartilage, muscles, etc. Moreover, the FTIR based scanning device 102 can be used for tissue measurements in laparoscopic surgeries. The FTIR based scanning device 102 can be used to characterize tissue in a healthy state, a disease state, and/or to detect and determine a response to treatment, for instance, by taking and comparing multiple measurements performed over a period of time.

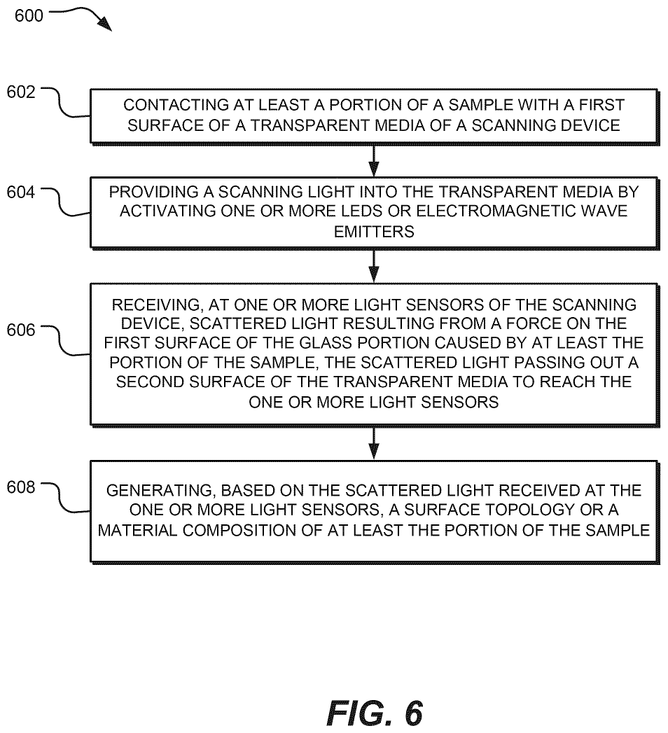

FIG. 6 depicts an example method 600 for performing a surface topology or composition analysis using an FTIR based scanning device 102.

In some examples, at operation 602, the method 600 contacts at least a portion of a sample with a first surface of a glass portion of a scanning device. At operation 604, the method 600 provides a scanning light into the glass portion by activating one or more LEDs. At operation 606, the method 600 receives, at one or more light sensors of the scanning device, scattered light resulting from a force on the first surface of the glass portion caused by at least the portion of the sample, the scattered light passing out a second surface of the glass portion to reach the one or more light sensors. At operation 608, the method generates, based on the scattered light received at the one or more light sensors, a surface topology or a material composition of at least the portion of the sample.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A frustrated total internal refraction (FTIR) based scanning device comprising:
a transparent media having a sample contact surface;
one or more electromagnetic wave emitters operable to provide a scanning light into the transparent media during a sample scanning procedure; and
one or more electromagnetic wave sensors, cameras, or microscopes, directed at a detection surface of the transparent media, operable to receive scattered light passing from the sample contact surface through the detection surface, the scattered light being used to represent a surface topology or a material composition of a sample contacting the sample contact surface during the sample scanning procedure.

2. The device of claim 1,
wherein,
the one or more electromagnetic wave emitters include a plurality of LEDs or electromagnetic wave emitters corresponding to a plurality of different wavelengths.

3. The device of claim 2, further comprising:
wherein,
providing the scanning light includes individually illuminating the plurality of LEDS to scan the sample with a sequence of different frequencies.

4. The device of claim 1,
wherein,
the transparent media includes a glass sheet, and
the one or more electromagnetic wave emitters are positioned at one or more side surfaces of the glass sheet for transmitting the scanning light into the glass sheet.

5. The device of claim 4,
wherein,
the glass sheet is a flat glass sheet or a curved glass sheet.

6. The device of claim 1, further comprising:
wherein,
the sample contact surface includes a raised portion operable to indent the sample during the sample scanning procedure.

7. The device of claim 1,
wherein,
the transparent media is formed into a handheld device with the sample contact surface defining an end of the handheld device, and
the one or more electromagnetic wave sensors, cameras, or microscopes are disposed in an interior portion of the handheld device.

8. The device of claim 1, further comprising:
a computing device having at least a display operable for presenting an image of a 3D topology generated from the scattered light.

9. The device of claim 8,
wherein,
the one or more electromagnetic wave sensors, cameras, or microscopes includes one or more of an infrared camera, a visible light camera, or an ultraviolet light camera.

10. A method to perform a surface topology or composition analysis, the method comprising:
contacting at least a portion of a sample with a first surface of a transparent media of a scanning device;
providing a scanning light into the transparent media by activating one or more LEDs or electromagnetic wave emitters;
receiving, at one or more light sensors of the scanning device, scattered light resulting from a force on the first surface of the transparent media caused by at least the portion of the sample, the scattered light passing out a second surface of the transparent media to reach the one or more light sensors; and
generating, based on the scattered light received at the one or more light sensors, a surface topology or a material composition of at least the portion of the sample.

11. The method of claim 10,
wherein,
the one or more LEDs or electromagnetic wave emitters include a plurality of different frequency LEDs or electromagnetic wave emitters; and
generating the surface topology or the material composition includes aggregating different frequencies of the scattered light, generated one-by-one by the plurality of different frequency LEDs or electromagnetic wave emitters into a profilometry for at least the portion of the sample contacting the first surface.

12. The method of claim 11,
wherein,
the one or more light sensors includes at least one of an infrared camera, a visible light camera, or an ultraviolet light camera disposed in the scanning device and directed at the transparent media.

13. The method of claim 10,
wherein,
providing the scanning light into the transparent media includes activating a plurality of LEDs or electromagnetic wave emitters positioned adjacent to a third surface of the transparent media.

14. The method of claim 13,
wherein,
the transparent media includes a glass sheet;
the first surface is an exposed top surface of the glass sheet;
the second surface is an unexposed bottom surface of the glass sheet opposite the exposed top surface; and
the third surface is a side surface of the glass sheet.

15. The method of claim 10,
wherein,
the force on the first surface of the transparent media caused by at least the portion of the sample creates the scattered light by using frustrated total internal refraction (FTIR).

16. A system for generating a surface topology or composition analysis, the system comprising:
a transparent media having a sample contact surface;
a plurality of LEDs with different frequencies operable to provide scanning light into a side of the transparent media during a sample scanning procedure;
one or more light sensors, directed at a detection surface of the transparent media and operable to receive scattered light resulting from a force at the sample contact surface; and
a surface topology or a material composition of a sample contacting the sample contact surface during the sample scanning procedure, the surface topology or the material composition being generated from the scattered light.

17. The system of claim 16 further comprising:
a profilometry of a portion of the sample contacting the sample contact surface during the sample scanning procedure, the profilometry including an aggregation of different frequencies of the scattered light, the surface topology being a three-dimensional representation of the profilometry.

18. The system of claim 16,
wherein,
the system is integrated into a handheld scanning device or a standing platform.

19. The system of claim 16,
wherein,
the system includes the material composition; and
the different frequencies are selectively activated to correspond to a target component of the material composition.

20. The system of claim 16,
wherein,
the sample includes a living body part of a human, a living body part of an animal, or a plant; or
the surface topology includes a tumor surface topography, a human organ surface topology, or a plant leaf surface topology.
